(19) United States
(12) Patent Application Publication
(10) Pub. No.: US 2025/0366897 A1
(43) Pub. Date: Dec. 4, 2025

(54) Adapter for a mixing system for preparing a bone cement paste and kit for preparing a bone cement paste comprising an adapter and a mixing system

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 19/222,542

(22) Filed: May 29, 2025

(30) Foreign Application Priority Data
Jun. 3, 2024 (EP) 24179486.6

(51) Int. Cl. A61B 17/88

(52) U.S. Cl. CPC A61B 17/8833; A61B 2017/8838

(57) ABSTRACT

An adapter for a mixing system for preparing a bone cement paste from two starting components, comprising a fastening element for reversibly fastening the adapter to a handle of the mixing system, wherein the handle is designed to mix the two starting components in a container of the mixing system by means of a mixing rod of the mixing system and to thus prepare the bone cement paste, and a connecting element for reversibly connecting the adapter to an electric drive device, wherein the adapter is designed to establish a force-transmitting connection between the electric drive device and the handle so that the mixing rod can be mechanically operated using the electric drive device.

### CROSS-REFERENCE TO RELATED APPLICATIONS

This application claims priority pursuant to 35 U.S.C. 119(a) to European Patent Application No. 24179486.6, filed Jun. 3, 2024, which application is incorporated herein by reference in its entirety.

### FIELD OF THE INVENTION

The invention relates to an adapter for a mixing system for preparing a bone cement paste from two starting components, comprising a fastening element for reversibly fastening the adapter to a handle of the mixing system, wherein the handle is designed to mix the two starting components in a container of the mixing system by means of a mixing rod of the mixing system and to thus prepare the bone cement paste, and a connecting element for reversibly connecting the adapter to an electric drive device, wherein the adapter is designed to establish a force-transmitting connection between the electric drive device and the handle so that the mixing rod can be mechanically operated using the electric drive device.

The invention further relates to a kit for preparing a bone cement paste from two starting components that comprises such an adapter and a mixing system, as well as to a method for preparing a bone cement paste from two starting components by means of such a kit.

### BACKGROUND OF THE INVENTION

Considerable efforts are being made to demonstrate devices and methods for providing bone cement by means of which bone cement paste can be provided simply, reliably, and quickly. An important aspect in the provision of bone cement paste is avoiding air inclusions, such as gas bubbles, in the bone cement. To avoid these, a plurality of vacuum cementing systems have been described, of which the following are mentioned by way of example: U.S. Pat. Nos. 6,033,105 A, 5,624,184 A, 4,671,263 A, 4,973,168 A, 5,100,241 A, WO 99/67015 A1, EP 1020167 A2, U.S. Pat. No. 5,586,821 A, EP 1016452 A2, DE 3640279 A1, WO 94/26403 A1, EP 1005901 A2, EP 1886647 A1, U.S. Pat. No. 5,344,232 A.

There is a desire in the market to simplify the provision of bone cement paste. A further development is the development of mixing systems in which both starting components are stored in separate regions of the mixing systems and are not mixed together in the mixing system itself until immediately before the cementing application. Such closed, so-called full-prepacked mixing systems are mentioned, for example, in the following documents: EP 0 692 229 A1, DE 10 2009 031 178 B3, U.S. Pat. Nos. 5,997,544 A, 6,709,149 B1, DE 698 12 726 T2, EP 0 796 653 A2, and U.S. Pat. No. 5,588,745 A.

In the aforementioned mixing systems, a monomer liquid is mechanically mixed with a bone cement powder, for example by means of a manually operable mixing rod.

The presence of a manually operable mixing rod integrated into the mixing system provides the user with the advantage that the bone cement paste is mixed and can be prepared without additional aids, such as a spatula. In addition, a mixing rod integrated into the mixing system minimizes application errors by the user and allows the bone cement paste to be prepared under the most sterile conditions possible.

However, depending on the consistency and/or quantity of the final desired bone cement paste, a hand-operated mixing rod can require a considerable amount of effort when mixing the starting components. Mechanical, in particular electrical, assistance when operating the mixing rod could make mixing the starting components easier for the user.

There is therefore a desire in the market for systems that are suitable for and/or can be modified for preparing a bone cement paste in the most simple, safe and at the same time flexible manner possible, either being operated manually or with mechanical, in particular electrical, assistance, depending on the preference of the user of the mixing system.

### SUMMARY OF THE INVENTION

The invention generally comprises an adapter for a mixing system for preparing a bone cement paste from two starting components, comprising a fastening element for reversibly fastening the adapter to a handle of the mixing system, wherein the handle is designed to mix the two starting components in a container of the mixing system by means of a mixing rod of the mixing system and to thus prepare the bone cement paste, and a connecting element for reversibly connecting the adapter to an electric drive device, wherein the adapter is designed to establish a force-transmitting connection between the electric drive device and the handle so that the mixing rod can be mechanically operated using the electric drive device, and a kit for preparing a bone cement paste from two starting components that comprises such an adapter and a mixing system, as well as to a method for preparing a bone cement paste from two starting components by means of such a kit.

One object of the present invention is to overcome, at least in part, one or more of the disadvantages resulting from the prior art.

In particular, the invention is based on the aim of providing an adapter for a mixing system for preparing a bone cement paste, which allows a simple, fast, use-safe and cost-effective connection between the mixing system and an electric drive device, by means of which the starting components of the bone cement paste can be mixed more easily.

In particular, the adapter is intended to allow the simplest and fastest possible connection between an electric drive device and a mixing rod of the mixing system, by means of which the starting components can be mixed in the mixing system. Preferably, the adapter is intended to be reversibly connectable to both the mixing system and the electric drive device.

A further object of the invention is to provide a kit for preparing a bone cement paste that comprises such an adapter and a mixing system, in which the starting components of the bone cement paste can be mixed by means of a mixing rod.

Another object of the invention is to provide a method by which bone cement can be prepared from two starting components, by means of which at least some of the above-described objects are at least partially achieved.

### PREFERRED EMBODIMENTS OF THE INVENTION

A contribution to the at least partial fulfillment of at least one of the aforementioned objects is made by the features of the independent claims. The dependent claims provide preferred embodiments that contribute to the at least partial fulfillment of at least one of the objects.

A first embodiment of the invention relates to an adapter for a mixing system for preparing a bone cement paste from two starting components, comprising a fastening element for reversibly fastening the adapter to a handle of the mixing system, wherein the handle is designed to mix the two starting components in a container of the mixing system by means of a mixing rod of the mixing system and to thus prepare the bone cement paste, and a connecting element for reversibly connecting the adapter to an electric drive device, wherein the adapter is designed to establish a force-transmitting connection between the electric drive device and the handle so that the mixing rod can be mechanically operated using the electric drive device.

In one embodiment of the adapter, the fastening element comprises a receptacle for receiving at least 20%, preferably at least 40%, most preferably at least 60%, of the surface area of the handle, based on the total surface area of the handle. This embodiment is a second embodiment of the invention, which is preferably dependent upon the first embodiment of the invention.

In one embodiment of the adapter, the receptacle has an inner geometry which is substantially complementary to an outer geometry of the handle in order to allow the handle to be substantially interlockingly connected inside the adapter. This embodiment is a third embodiment of the invention, which is preferably dependent upon the second embodiment of the invention.

In one embodiment of the adapter, the adapter comprises a reversibly detachable closure cap which is designed to fix the handle within the receptacle by at least partially covering the receptacle. This embodiment is a fourth embodiment of the invention, which is preferably dependent on the second to fourth embodiments of the invention.

In one embodiment of the adapter, the closure cap can be reversibly detachably fastened to the receptacle by locking, clamping, a cotter pin and/or a hook-and-loop fastener in order to at least partially cover the receptacle. This embodiment is a fifth embodiment of the invention, which is preferably dependent on the fourth embodiment of the invention.

In one embodiment of the adapter, the connecting element is arranged at an end of the adapter that is axially opposite the fastening element. This embodiment is a sixth embodiment of the invention, which is preferably dependent on any one of the preceding embodiments of the invention.

In one embodiment of the adapter, the connecting element comprises a rod with, in a radial cross section, a round, square, or preferably hexagonal, cross section. This embodiment is a seventh embodiment of the invention, which is preferably dependent on any one of the preceding embodiments of the invention.

In one embodiment of the adapter, the adapter is designed such that, when the adapter is fastened to the handle of the mixing system, the mixing rod of the mixing system has a common longitudinal axis with the connecting element of the adapter.

This embodiment is an eighth embodiment of the invention, which is preferably dependent on any of the previous embodiments of the invention.

A ninth embodiment of the invention relates to a kit for preparing a bone cement paste from two starting components that comprises an adapter according to any of the first to eighth embodiments of the invention and a mixing system, wherein the mixing system comprises a container in which a bone cement powder can, or preferably is, stored as a first starting component, and a mixing rod which is axially displaceable and rotatably mounted in the container and comprises a handle which is accessible from outside the container in order to rotate the mixing rod inside the container and thus prepare the bone cement paste.

In one embodiment of the kit, the mixing system comprises a reservoir in which a monomer liquid can, or preferably is, stored as a second starting component. This embodiment is a tenth embodiment of the invention, which is preferably dependent on the ninth embodiment of the invention.

An eleventh embodiment of the invention is a method for preparing a bone cement paste from two starting components by means of a kit according to either the ninth or tenth embodiment of the invention, comprising the following method steps:
a. providing the two starting components in the container;
b. fastening the adapter to the handle using the fastening element;
c. connecting the adapter to an electric drive device using the connecting element; and,
d. mixing the starting components in the container by means of the mixing rod by actuating the electric drive device, wherein the bone cement paste is prepared by mixing.

One embodiment of the method comprises a method step e., wherein method step e. involves detaching the adapter from the handle of the mixing rod. This embodiment is a twelfth embodiment of the invention, which is preferably dependent upon the eleventh embodiment of the invention.

One embodiment of the method comprises a method step f., wherein method step f. involves removing the bone cement paste from the mixing system. This embodiment is a thirteenth embodiment of the invention, which is preferably dependent on the eleventh or twelfth embodiment of the invention.

### General

In the present description, specifications of ranges also contain the values specified as limits. A specification of the type “in the range from X to Y” with respect to a quantity A consequently means that A can take the values X, Y and values between X and Y. Ranges which are limited on one side, of the type “up to Y” for a size A, accordingly mean a value Y and less than Y.

Some of the features described are associated with the term “substantially.” The term “substantially” is to be understood in such a way that, under real conditions and manufacturing techniques, a mathematically exact interpretation of terms such as “superimposition,” “perpendicular,” “diameter” or “parallelism” can never be given exactly, but only within certain manufacturing error tolerances. For example, “substantially perpendicular axes” enclose an angle of 85 degrees to 95 degrees relative to one another, and “substantially equal volumes” comprise a variation of up to 5% by volume. For example, a “device consisting substantially of plastics” comprises a plastics content of ≥95 to ≤100% by weight. For example, a “substantially complete filling of a volume B” comprises a filling of ≥95 to ≤100% by volume of the total volume of B.

The terms “proximal” and “distal” merely serve to designate the spatially opposite ends of the device or other structural units of the device, and do not allow any conclusions to be drawn about the orientation in relation to a human body, for example a user of the device. “Distal to . . . ” and “proximal to . . . ” or similar formulations merely express the spatial arrangement of two structural units of the device in relation to one another.

### DETAILED DESCRIPTION

The invention firstly relates to an adapter for a mixing system for preparing a bone cement paste from two starting components, comprising a fastening element for reversibly fastening the adapter to a handle of the mixing system, wherein the handle is designed to mix the two starting components in a container of the mixing system by means of a mixing rod of the mixing system and to thus prepare the bone cement paste, and a connecting element for reversibly connecting the adapter to an electric drive device, wherein the adapter is designed to establish a force-transmitting connection between the electric drive device and the handle so that the mixing rod can be mechanically operated using the electric drive device.

The adapter is used to temporarily and reversibly connect a mixing system for preparing a bone cement paste from two starting components to an electric drive device, such as a type of electric drill, in a force-transmitting manner such that the electric drive device can transmit a rotational movement to a mixing rod of the mixing system via the adapter. The mixing rod rotating in this way serves to mix the starting components in the mixing system, in particular in a container of the mixing system, thereby preparing the bone cement paste.

The adapter thus gives a user of the mixing system the option of operating it both manually, i.e., using physical force to mix the bone cement paste, and with the aid of an electric drive device. The adapter thus simply, inexpensively and rapidly increases the flexibility for the user of the mixing system.

The adapter is designed to be reversibly fastened to a handle of a mixing system by means of a fastening element, wherein the handle is in turn directly or indirectly connected to a mixing rod. Rotating the handle thus leads to a rotational movement of the mixing rod, which can mix the starting components in the mixing system to form a bone cement paste.

A handle of a mixing system makes it easier for the user to operate a mixing rod of the mixing system, as it is designed to be clasped by the user's hand, thus making it easier to rotate the mixing rod. A handle is a component that can be securely gripped by a hand in order to rotate the mixing rod and preferably also to move it axially in the mixing system. Preferably, the handle is ergonomically shaped so that it can be easily and securely grasped by a hand.

For the temporary, reversible connection of the adapter to an electrical drive device, such as a type of drill, the adapter comprises a connecting element.

The adapter thus comprises two elements, the fastening element and the connecting element, in order to reversibly connect a mixing system to an electric drive device in such a way that a rotational movement of the electric drive device is transmitted via the adapter to a mixing rod of the mixing system, in particular directly and immediately.

The fastening element can have a different shape so as to be reversibly fastened to a handle of a mixing system.

One embodiment of the adapter is characterized in that the fastening element comprises a receptacle for receiving at least 20%, preferably at least 40%, most preferably at least 60%, of the surface area of the handle, based on the total surface area of the handle.

A receptacle is a structured and/or shaped recess which is suitable for receiving the handle therein and at least partially enclosing it. The handle can thus be inserted into the receptacle.

Preferably, the entire handle can be received in the receptacle of the fastening element.

The receptacle thus encloses at least part, preferably at least 20%, preferably at least 40%, most preferably at least 60%, of the surface area of the handle, based on the total surface area of the handle. The receptacle thus imitates a hand of a user of the mixing system, ensuring that the adapter is securely fastened to the mixing system.

One embodiment of the adapter is characterized in that the receptacle has an inner geometry which is substantially complementary to an outer geometry of the handle of the mixing system in order to enable the handle to be substantially interlockingly connected inside the adapter.

As a result, the adapter ensures that the mixing rod is rotated in the most direct and controllable manner possible by means of an electric drive device.

The adapter can have a different shape so as to securely and reversibly fasten a mixing rod in the receptacle.

One embodiment of the adapter is characterized in that it comprises a reversibly detachable closure cap which is designed to fix the handle within the receptacle by at least partially covering the receptacle.

When the closure cap is open, the handle can be inserted into the holder and the cap can then be closed, fixing the handle in the receptacle. When closed, the closure cap covers an opening of the receptacle, through which the handle can be inserted into the receptacle, such that the handle can no longer be removed from the receptacle through the opening without opening the closure cap. The closure cap is therefore a simple way of mechanically fixing the handle in the receptacle.

Preferably, the receptacle and the closure cap are formed in two parts.

More preferably, the adapter is formed in two parts, wherein the fastening element and the receptacle are integrally formed and form the first part of the two-part adapter and the closure cap forms the second part of the two-part adapter.

The closure cap can have a different shape so as to be reversibly fastenable to the receptacle in such a way that the receptacle can be at least partially covered and the handle can thus be fixed in the receptacle.

One embodiment of the adapter is characterized in that the closure cap can be reversibly detachably fastened to the receptacle by locking, by clamping, by a cotter pin and/or by a hook-and-loop fastener in order to at least partially cover the receptacle, in particular the opening of the receptacle. These different types of fastening options allow for easy and, in particular, fast fastening of the adapter to the handle of the mixing system, which is particularly advantageous during operations.

One embodiment of the adapter is characterized in that the connecting element is arranged at an end of the adapter that is axially opposite the fastening element. Preferably, the connecting element and the fastening element are arranged on a common axis, in particular a longitudinal axis of the adapter. This allows the adapter to transmit force from an electric drive device to the mixing system as efficiently as possible. This also makes the adapter easier to use.

The connecting element can have a different design so as to be reversibly connectable to an electric drive device.

Preferably, the adapter is designed to reversibly connect a handle of a mixing system to an electric drive device comprising a chuck, preferably a three-jaw chuck, which is preferably coupled to a rotary spindle. In such a system, a rotational movement, preferably performed via the rotary spindle, can be intended to be transmitted to the mixing system via the chuck and the adapter.

One embodiment of the adapter is characterized in that the connecting element comprises a rod with, in a radial cross section, a round, square, or preferably hexagonal, cross section. Such a rod can be clamped quickly, easily and securely in a chuck of an electric drive device, allowing for a rotational movement of a rotary spindle via the chuck and the clamped adapter and ultimately to a mixing rod for mixing a bone cement paste.

One embodiment of the adapter is characterized in that the adapter is designed such that when the adapter is fastened to the handle of a mixing system, the mixing rod of the mixing system has a common axis, in particular a longitudinal axis, with the connecting element of the adapter. In this embodiment, the adapter acts as a kind of extension of the mixing rod, wherein a user can use the fastening means to rotate the mixing rod instead of the handle of the mixing system. Furthermore, this design allows for efficient and easy rotation of the mixing rod via an electric drive device.

The invention further relates to a kit for preparing a bone cement paste from two starting components. The kit comprises an adapter according to any one of the aforementioned embodiments and a mixing system.

The mixing system comprises a container in which a bone cement powder can, or preferably is, stored as a first starting component, and a mixing rod with a handle that is reversibly axially displaceable and rotatably mounted in the container, in particular mounted so as to reversibly rotate about its own longitudinal axis. The handle is located outside the container and is designed to rotate the mixing rod inside the container and preferably also to move it axially inside the container, thus preparing the bone cement paste.

The features of a mixing system described in connection with the adapter also apply to the mixing system of the kit.

One embodiment of the kit is characterized in that the mixing system comprises a reservoir in which a monomer liquid can, or preferably is, stored as a second starting component. Preferably, the monomer liquid is located in a monomer liquid container.

A monomer liquid container is a vessel for the monomer liquid in which said liquid can be stored safely and, in particular, sterilely until it is used. Different monomer liquid containers are known to a person skilled in the art.

For example, the monomer liquid container can be a bag. Preferred bags are multilayer composite films with an EVOH (ethylene vinyl alcohol) barrier layer, optionally comprising a metal coating, in particular comprising an aluminum coating.

Preferably, the monomer liquid container is an ampoule, preferably a glass ampoule. Ampoules are preferred because they can be opened with greater control and it is easier for the entire monomer liquid to flow out of ampoules that open in a fluid-conducting manner.

Preferably, both the bone cement powder (in the container) and at least one monomer liquid container (in the reservoir) are stored in the mixing system. This makes it easier for a user to use the kit, in particular it allows a bone cement paste to be prepared under sterile conditions.

The invention also relates to a method for preparing a bone cement paste from two starting components by means of a kit according to any one of the preceding embodiments, comprising the following method steps:
a. providing the two starting components in the container;
b. fastening the adapter to the handle using the fastening element;
c. connecting the adapter to an electric drive device using the connecting element; and,
d. mixing the starting components in the container by means of the mixing rod by actuating the electric drive device, wherein the bone cement paste is prepared by mixing.

In method step a., the two starting components of the bone cement paste are provided in the container of the kit's mixing system in order to be mixed. For this purpose, the monomer liquid is preferably conveyed as the second starting component from the reservoir of the mixing system and into the container of the mixing system, in which the bone cement powder is already stored as the first starting component.

In method step b., the adapter is fastened to the handle of the mixing system using the fastening element.

In a preferred embodiment of method step b., for this to happen the handle is inserted into the receptacle of the fastening element, which is preferably substantially complementary to the outer geometry of the handle, and fixes the receptacle by the reversibly detachable closure cap by partially covering the receptacle. Preferably, at least 60% of the surface area of the handle based on the total surface area of the handle is received in the receptacle.

In method step c., the adapter is connected to an electric drive device by means of the connecting element.

In a preferred embodiment of method step c., the connecting element in the form of a rod with a hexagonal radial cross section is clamped in a chuck of the electric drive device.

Method steps a., b. and c. can be carried out in any order. Preferably, however, the adapter is at least connected to the electric drive device before the monomer liquid is conveyed into the bone cement powder.

In method step d., the starting components are mixed in the mixing system to prepare the bone cement paste. For this purpose, the electric drive device is appropriately put into operation, whereby a rotational movement of the drive device is transmitted via the adapter, preferably directly, to the handle of the mixing system. By turning the handle, the mixing rod of the mixing system rotates, preferably directly, causing the starting components to mix in the mixing system.

One embodiment of the method is characterized in that, in one method step, step e., the adapter is detached from the handle of the mixing rod. The method step is preferably carried out after method step d. By detaching the adapter from the handle, the electric drive device and the mixing system are separated again and a user of the kit can continue to use the mixing system as if the bone cement paste had been mixed by manually operating the mixing rod. The adapter is therefore immediately available again for use with another mixing system, preferably of the same design, for example.

One embodiment of the method is characterized in that, in one method step, step f., the bone cement paste is removed from the mixing system. Method step f. takes place after method step d. and preferably, if used, also after method step e.

The kit and/or mixing system is/are characterized by the fact that they prepare a bone cement paste consisting of two starting components. A bone cement paste is a substance which is suitable in the field of medical technology for creating a stable connection between artificial joints, such as hip and knee joints, and bone material. Curing turns bone cement paste into bone cement. These bone cements are preferably polymethylmethacrylate bone cements (PMMA bone cements). PMMA bone cements have been used in medical applications for a long time, and go back to the work of Sir Charnley (cf. Charnley, J., Anchorage of the femoral head test of the drawing of the femur, J. Bone Joint Surg. 1960 (42) 28-30.). PMMA bone cements can be produced from a bone cement powder as the first starting component and a monomer liquid as the second starting component. With a suitable composition, the two starting components can be stably stored separately from one another. When the two starting components are brought into contact, the swelling of the polymer components of the bone cement powder produces a plastically deformable bone cement paste. A polymerization of the monomer is initiated by radicals. As the polymerization of the monomer progresses, the viscosity of the bone cement paste increases until it cures completely.

A bone cement powder is a powder which comprises at least one particulate polymethyl methacrylate and/or a particulate polymethylmethacrylate copolymer. Examples of copolymers are styrene and/or methyl acrylate. In one embodiment, the bone cement powder can additionally comprise a hydrophilic additive which supports the distribution of the monomer liquid within the bone cement powder. In a further embodiment, the bone cement powder can additionally comprise an initiator which initiates the polymerization. In a further embodiment, the bone cement powder can additionally comprise a radiopaque. In yet a further embodiment, the bone cement powder can additionally comprise pharmaceutically active substances, for example antibiotics.

Preferably, the bone cement powder comprises, as the hydrophilic additive, at least one particulate polymethyl methacrylate and/or a particulate polymethyl methacrylate copolymer, an initiator and a radiopaque, or consists of these components. Further preferably, the bone cement powder comprises at least one particulate polymethyl methacrylate and/or a particulate polymethylmethacrylate copolymer, an initiator, a radiopaque, and a hydrophilic additive, or consists of these components. Most preferably, the bone cement powder comprises at least one particulate polymethyl methacrylate and/or a particulate polymethylmethacrylate copolymer, an initiator, a radiopaque, a hydrophilic additive, and an antibiotic, or consists of these components.

According to the invention, the particle size of the particulate polymethyl methacrylate and/or of the particulate polymethylmethacrylate copolymer of the bone cement powder can correspond to the sieve fraction of less than 150 μm, preferably less than 100 μm.

According to the invention, the hydrophilic additive can be particulate and/or fibrous. In a further embodiment, the hydrophilic additive can be sparingly soluble, preferably insoluble, in methyl methacrylate. In a further embodiment, the hydrophilic additive can have an absorption capacity of at least 0.6 g of methyl methacrylate per gram of hydrophilic additive. In a further embodiment, the hydrophilic additive can have a chemical substance having at least one OH group. It can preferably be provided that the hydrophilic additive has covalently bonded OH groups on its surface. Examples of such preferred hydrophilic additives can be additives selected from the group comprising cellulose, oxycellulose, starch, titanium dioxide, and silicon dioxide, wherein fumed silicon dioxide is particularly preferred. In one embodiment, the particle size of the hydrophilic additive can correspond to the sieve fraction of less than 100 μm, preferably less than 50 μm and most preferably less than 10 μm. The hydrophilic additive can be contained in an amount of 0.1 to 2.5 wt. % relative to the total weight of the bone cement powder.

According to the invention, the initiator can contain dibenzoyl peroxide or consist of dibenzoyl peroxide.

According to the invention, a radiopaque is understood to be a substance which makes it possible to make the bone cement visible on X-ray diagnostic images. Examples of radiopaques can include barium sulfate, zirconium dioxide, and calcium carbonate.

According to the invention, the pharmaceutically active substance can comprise one or more antibiotics and optionally added co-factors for the one or more antibiotics. Preferably, the pharmaceutically active substance consists of one or more antibiotics and optionally added co-factors for the one or more antibiotics. Examples of antibiotics include, inter alia, gentamicin, clindamycin, and vancomycin.

According to the invention, the monomer liquid can comprise the monomer methyl methacrylate or consist of methyl methacrylate. In one embodiment, the monomer liquid comprises, in addition to the monomer, an activator dissolved therein, such as N,N-dimethyl-p-toluidine, or consists of methyl methacrylate and N,N-dimethyl-p-toluidine.

The features disclosed in connection with the adapter are also disclosed for the kit, the mixing system and the method, and vice versa.

### BRIEF DESCRIPTION OF THE DRAWINGS

he invention is further illustrated by way of example below by means of figures. The invention is not limited to the figures, wherein:

FIG. 1 is a perspective side view of an example kit for preparing a bone cement paste that comprises an adapter and a mixing system, and a perspective side view of an electric drive device;

FIG. 2 is a schematic longitudinal section through the components of the kit and the electric drive device in FIG. 1;

FIG. 3 is a schematic longitudinal section and a schematic side view of the adapter from FIGS. 1 and 2, each with an open and a closed closure cap;

FIG. 4 is a schematic longitudinal section of the kit and the electric drive device from the previous figures, wherein the adapter is connected to the electric drive device via a connecting element;

FIG. 5 is a schematic longitudinal section of the kit and the electric drive device from the previous figures, wherein the adapter is connected to the electric drive device via the connecting element and to a handle of the mixing system via a fastening element;

FIG. 6 is a perspective side view of the kit and the electric drive device from the previous figures, wherein the adapter is connected to the electric drive device via the connecting element and to the handle of the mixing system via the fastening element;

FIG. 7 is a schematic side view of another example kit for preparing a bone cement paste that comprises an adapter and a mixing system, and,

FIG. 8 is a flowchart of a method for producing a bone cement paste.

### DETAILED DESCRIPTION OF THE INVENTION

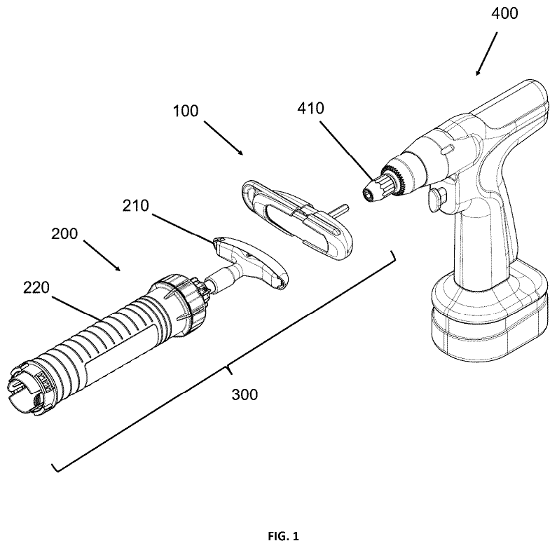

FIG. 1 is a perspective side view of an example kit 300 for preparing a bone cement paste from two starting components as well as a perspective side view of an example electric drive device 400. The kit 300 comprises an adapter 100 and a mixing system 200 comprising a handle 210. The adapter 100 is designed to be connected both to the handle 210 of the mixing system 200 and to a chuck 410 of the electric drive device 400 such that a force-transmitting connection is formed between the handle 310 and the electric drive device 400 so that a rotational movement of the electric drive device 400 can be transmitted via the adapter 100 to the handle 210, allowing a bone cement paste to be prepared in a container 220 of the mixing system 200. The functionality will be described in more detail in connection with the following figures.

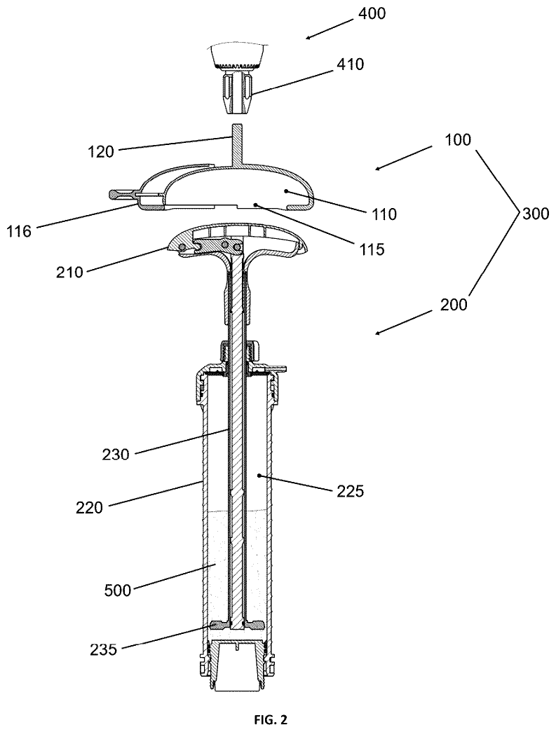

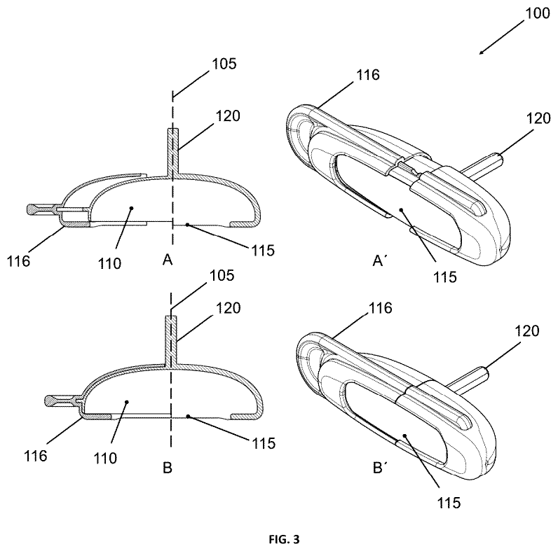

FIG. 2 is a schematic longitudinal section of the kit 300 and a portion of the electric drive device 400 from FIG. 1. The adapter 100 comprises a fastening element 110 in the form of a receptacle for receiving the handle 210 of the mixing system 200. The receptacle 110 is shaped to completely receive the handle 210 and has an inner geometry that is substantially complementary to an outer geometry of the handle. The embodiment shown thus enables the handle 210 to be substantially interlockingly connected inside the adapter 100, in particular inside the fastening element 110 of the adapter 100. In order to fix the handle 210 in the fastening element 110 after it has been inserted, the adapter 100 comprises a reversibly detachable closure cap 116, which is open in FIG. 2. When the closure cap 116 is open, the handle 210 can be inserted into and removed from the fastening element 110. Reference is made to the following figures, in particular FIG. 3, with regard to the function of the closure cap 116.

At an end that is axially opposite the fastening element 110, the adapter 100 comprises a connecting element 120 in the form of a rod, in particular a hexagonal rod (cf. FIG. 3). The fastening element 120 can be inserted into the chuck 410 of the electric drive device 400 and fixed therein.

FIG. 2 shows that the handle 210 is connected to a mixing rod 230 which projects into an interior 225 of the container 220 of the mixing system 200 where it is rotatably and axially displaceably mounted. The mixing rod 230 comprises, at an end that is axially opposite the handle 210, a mixing element 235 in the form of radially projecting blades, which facilitate mixing of the starting components of the bone cement paste.

A bone cement powder 500 is stored as the first starting component of the bone cement paste in the interior 225.

FIG. 3 shows the adapter 100 from FIGS. 1 and 2 both in a schematic longitudinal section (3A and 3B) and in a perspective side view (3A′ and 3B′).

The closure cap 116 is open in FIG. 3A and 3A′, as already also shown in FIG. 2. When open, the closure cap 116 is displaced radially outward from a longitudinal axis 105 of the adapter 100, which extends through the connecting element 120. As a result, the opening 115 of the fastening element 110 is released such that the handle 410 of the mixing system 200 (cf., for example, FIG. 2) can be inserted into the adapter 100 in order to be fixed therein in the fastening element 110.

In FIG. 3B and 3B′, however, the closure cap 116 is closed. The closure cap 116 can be reversibly switched from the open to the closed state by moving it in the direction of the longitudinal axis 105. When closed, the closure cap 116 can fix the handle 210 of the mixing system 200 in the fastening element 110 so that a rotational movement exerted on the connecting element 120 can be transmitted to the handle 210 via the fastening element 110. For this purpose, the closure cap 116 covers part of the opening 115 of the fastening element 110, which prevents the handle 210 arranged in the fastening element 110 from being removed therefrom. In order to reliably secure the closure cap 116 in the closed state, it is reversibly fastened to the fastening element 110 by locking.

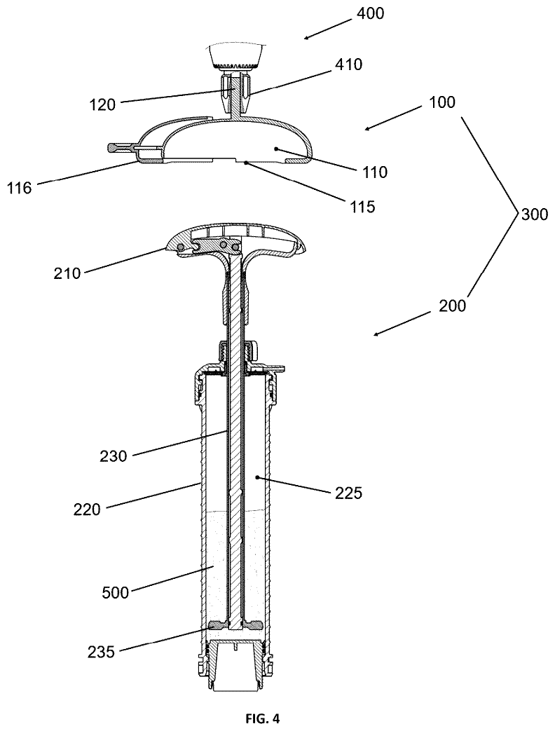

FIG. 4 is a schematic longitudinal section of the kit 300 and the electric drive device from FIGS. 1 and 2, wherein the connecting element 120 is inserted into the chuck 410 of the electric drive device 400 where it is fixed. The closure cap 116 is, as already also shown in FIG. 2, in the open state such that the handle 410 can be inserted into the fastening element 110.

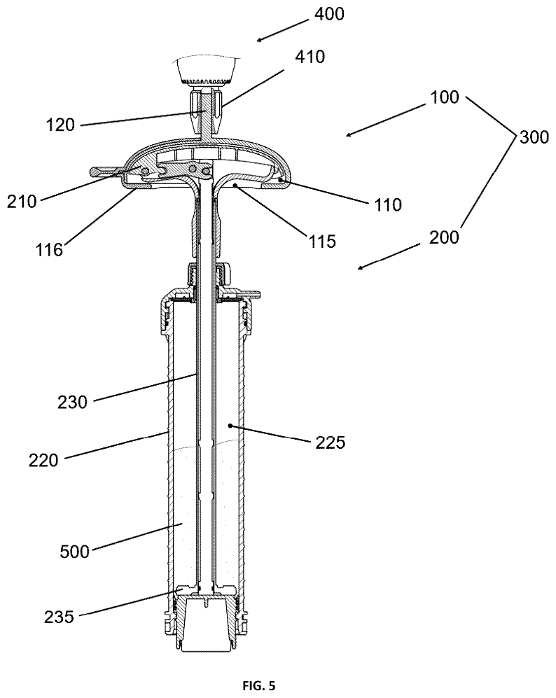

FIG. 5 is a schematic longitudinal section of the kit 300 and the electric drive device from FIGS. 1, 2 and 4, wherein, in addition to FIG. 4, the handle 210 of the mixing system 200 has been fixed in the fastening means 110. For this purpose, the closure cap 116 was moved in the direction of the longitudinal axis 105 of the adapter 100 (cf. FIG. 3), as a result of which it was locked to the fastening means 110 in the closed state and thus fastened. The adapter 100 in FIG. 5 thus allows for a force-transmitting connection between the electric drive device 400, in particular the chuck 410, and the handle 210 of the mixing system 200. A rotational movement triggered by appropriate actuation of the electric drive device 400 is thus transferred directly to the mixing rod 230, which allows the bone cement paste to be mixed during its preparation. In FIG. 5, only the provision of a monomer liquid as a second starting component in the interior 225 of the container 220 is missing.

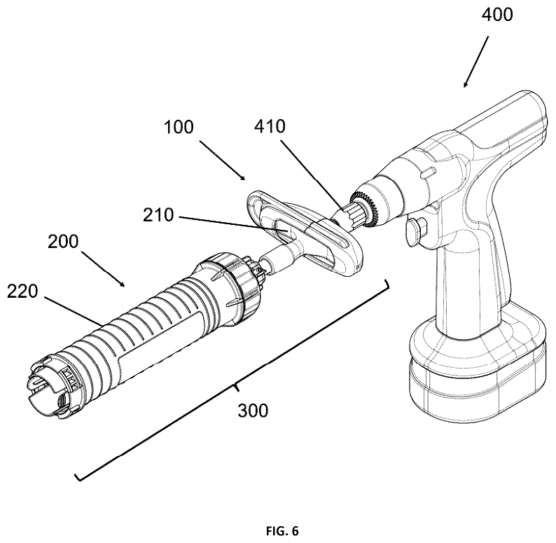

FIG. 6 is a perspective side view of the kit 300 and the electric drive device from FIGS. 1, 2, 4 and 5, wherein, as described in respect of FIG. 5, the adapter 100 is connected to both the electric drive device 400 and the handle 210 and has thus established a force-transmitting connection between the two. By appropriately actuating the electric drive device 400, the starting components of the bone cement paste can thus be mixed in the mixing system 200, in particular in the interior 225 (cf., for example, FIG. 2) of the container 220, and the bone cement paste can thereby be prepared.

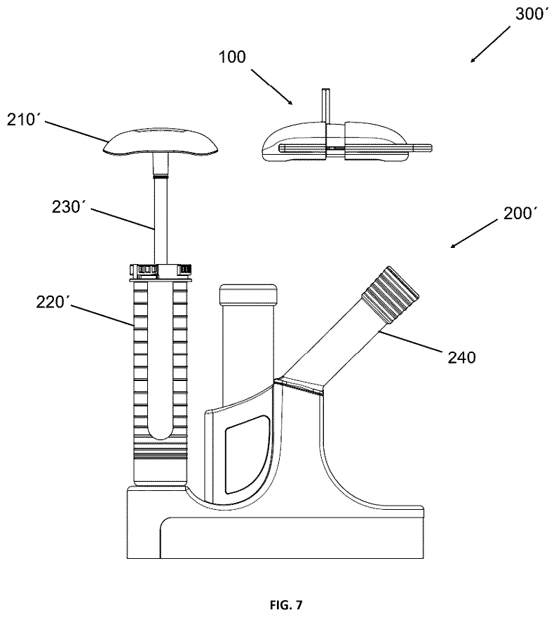

FIG. 7 is a schematic side view of another example kit 300′ for preparing a bone cement paste from two starting components. The kit 300′ comprises an adapter 100, as already described in the previous figures, and another example mixing system 200′. The mixing system 200′ differs from the mixing system 200 in the previous figures by an additional reservoir 240 in which a glass ampoule (not shown) filled with a monomer liquid as the second starting component is stored. Furthermore, a bone cement powder is stored in a container 210′ of the mixing system 200′ as the first starting component (not shown). For the exact structure and function of the mixing system 200′, reference is hereby made to the disclosure of application EP3093067A1.

With regard to the use of the adapter 100 with the mixing system 200′, in particular with its handle 210′, reference is made to the previous figures, since the mixing system 200′ does not differ significantly from the mixing system 200 in the previous figures in terms of the key points. The adapter 100 can therefore be used with the mixing system 200′ in a manner substantially analogously to the figures described above.

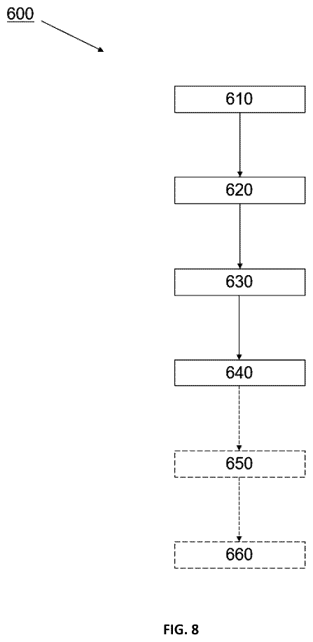

FIG. 8 shows an example method 600 for preparing a bone cement paste from two starting components by means of a kit 300, 300′ from the previous figures, comprising method steps 610 to 640 and optionally method step 650 and/or method step 660, wherein the order of method steps 610 to 630 can also differ from that shown.

In a method step 610, the two starting components are provided in the container 210, 210′, in particular in the interior 225 of the container 210, 210′, of the mixing system 200, 200′. Preferably, the bone cement powder 500 is already stored in the container 210, 210′ as the first starting component of the bone cement paste before the monomer liquid is conveyed into the container 210, 210′ as the second starting component. The monomer liquid is preferably stored in a reservoir 240 of the mixing system 200′ before being conveyed into the container 210, 210′.

In a method step 620, the adapter 100 is fastened by means of the fastening element 110 to the handle 210, 210′ of the mixing system 200, 200′. For this purpose, the handle 210, 210′ is preferably inserted into a receptacle of the fastening element 110 where it is fixed by means of a closure cap 116.

In a method step 630, the adapter 100 is connected by means of the connecting element 120, preferably in the form of a rod, preferably a hexagonal rod, to an electric drive device 400, preferably to a chuck 410 of an electric drive device.

By means of method steps 620 and 630, a force-transmitting connection is established between the electric drive device 400 and the handle 210, 210′ so that the mixing rod 230, 230′ can be mechanically operated using the electric drive device.

In a method step 640, the starting components of the bone cement paste prepared in the container in method step 610 are mixed. For this purpose, the electric drive device 400 is appropriately actuated, for example by pressing a power button of the electric drive device 400, whereby a rotational movement of the electric drive device 400 is transmitted to the mixing rod 230, of the mixing system 200, 200′ via the adapter 100 and the handle 210, 210′. The mixing rod 230, 230′ thus driven by the electric drive device 400 mixes the starting components provided to prepare the bone cement paste.

In an optional method step, step 650, the adapter 100 can be detached from the handle 210, 210′ of the mixing system 200, 200′ again. This allows the handle 210, 210′ to continue to be operated manually and/or the adapter 100 to continue to be used with another mixing system 200, 200′.

In an optional method step, step 660, the bone cement paste prepared during the method 600 is removed from the mixing system 200, 200′. For this purpose, method step 650 is preferably carried out first.

### REFERENCE SIGNS

100 Adapter

110 Fastening element

115 Opening

116 Closure cap

120 Connecting element

200, 200′ Mixing system

210, 210′ Handle

220, 220′ Container

225 Interior

230, 230′ Mixing rod

235 Mixing element

240 Reservoir

300, 300′ Kit

400 Electric drive device

410 Chuck

500 Bone cement powder

600 Method

610 Providing

620 Fastening

630 Connecting

640 Mixing

650 Detaching

660 Removing

What is claimed is:

1. An adapter (100) for a mixing system (200, 200′) for preparing a bone cement paste from two starting components, comprising:
a fastening element (110) for reversibly fastening the adapter (100) to a handle (210, 210′) of the mixing system (200, 200′), wherein the handle (210, 210′) is designed to mix the two starting components in a container (220, 220′) of the mixing system (200, 200′) by means of a mixing rod (230, 230′) of the mixing system (200, 200′) and to thus prepare the bone cement paste, and,
a connecting element (120) for reversibly connecting the adapter (100) to an electric drive device (400), wherein the adapter (100) is designed to establish a force-transmitting connection between the electric drive device (400) and the handle (210, 210′) so that the mixing rod (230, 230′) can be mechanically operated using the electric drive device (400).

2. The adapter (100) according to claim 1, wherein the fastening element (110) comprises a receptacle for receiving at least 20%, preferably at least 40%, most preferably at least 60%, of the surface area of the handle (210, 210′), based on the total surface area of the handle (210, 210′).

3. The adapter (100) according to claim 2, wherein the receptacle has an inner geometry which is substantially complementary to an outer geometry of the handle (210, 210′) in order to allow the handle (210, 210′) to substantially interlockingly connect inside the adapter (100).

4. The adapter (100) according to any of claim 2, comprising a reversibly detachable closure cap (116) which is designed to fix the handle (210, 210′) within the receptacle by at least partially covering the receptacle.

5. The adapter (100) according to claim 4, wherein the closure cap (116) can be reversibly detachably fastened to the receptacle by locking, by clamping, by a cotter pin and/or by a hook-and-loop fastener in order to at least partially cover the receptacle.

6. The adapter (100) according to claim 1, wherein the connecting element (120) is arranged at an end of the adapter (100) that is axially opposite the fastening element (110).

7. The adapter (100) according to claim 1, wherein the connecting element (120) comprises a rod with, in a radial cross section, a round, square, or preferably hexagonal, cross section.

8. The adapter (100) according to claim 1, wherein the adapter (100) is designed such that, when the adapter (100) is fastened to the handle (210, 210′) of the mixing system (200, 200′), the mixing rod (210, 210′) of the mixing system (200, 200′) has a common longitudinal axis with the connecting element (120) of the adapter (100).

9. A kit (300, 300′) for preparing a bone cement paste from two starting components, comprising an adapter (100) according to claim 1 and a mixing system (200, 200′), wherein the mixing system (200, 200′) comprises a container (220, 220′) in which a bone cement powder (500) is or can be stored as a first starting component, and, a mixing rod (230, 230′) which is axially displaceable and rotatably mounted in the container (220, 220′) and comprises a handle (210, 210′) that is accessible from outside the container (220, 220′) in order to rotate the mixing rod (230, 230′) inside the container (220, 220′) and thus prepare the bone cement paste.

10. The kit (300′) according to claim 9, wherein the mixing system (200′) comprises a reservoir (240) in which a monomer liquid is or can be stored as a second starting component.

11. A method (600) for preparing a bone cement paste consisting of two starting components by means of a kit (300, 300′) according to either claim 9, comprising the following method steps:
a. providing (610) the two starting components in the container (220, 220′);
b. fastening (620) the adapter (100) to the handle (210, 210′) using the fastening element (110);
c. connecting (630) the adapter (100) to an electric drive device (400) using the connecting element (120);
d. mixing (640) the starting components in the container (220, 220′) by means of the mixing rod (230, 230′) by actuating the electric drive device (400), wherein the bone cement paste is prepared by mixing (640).

12. The method (600) according to claim 11, comprising a method step e., wherein method step e. involves detaching (650) the adapter (100) from the handle (210, 210′) of the mixing rod (230, 230′).

13. The method (600) according to claim 11, comprising a method step f., wherein method step f. involves removing (660) the bone cement paste from the mixing system (200, 200′).
